(19) United States
(12) Patent Application Publication
(10) Pub. No.: US 2023/0381589 A1
(43) Pub. Date: Nov. 30, 2023

(54) SYSTEM AND METHOD FOR EVALUATING USER PERFORMANCE ACROSS DIFFERENT ACTIVITIES

(71) Applicant: Orpyx Medical Technologies Inc., Calgary (CA)

(72) Inventors: CORY DAVID PADDOCK, CALGARY (CA); JEFFREY THOMPSON KNIGHT, AUSTIN, TX (US)

(21) Appl. No.: 18/316,350

(22) Filed: May 12, 2023

Related U.S. Application Data
(60) Provisional application No. 63/345,139, filed on May 24, 2022.

(51) Int. Cl. A63B 24/00; G01C 21/16

(52) U.S. Cl. CPC A63B 24/0062; G01C 21/16; A63B 2220/836; A63B 2220/51; A63B 2230/75; A63B 2024/0071

(57) ABSTRACT

A system, method, and computer program product for evaluating user performance across different activities is provided. In some examples, sensor data is obtained from sensors associated with a user while the user is performing one or more activities. The sensor data may be obtained from the user while the user performs activities corresponding to different activity categories, including gaming and non-gaming activities. The value of a standard athletic metric can be generated for the user for each activity using the corresponding sensor data. An activity category can also be determined for each activity. A user performance score for a given activity can be generated based on the corresponding standard athletic metric value and activity category.

### CROSS-REFERENCE TO RELATED APPLICATIONS

This application claims the benefit of priority of U.S. Provisional Application No. 63/345,139 filed May 24, 2022, which is incorporated herein by reference.

### FIELD

This document relates to systems and methods for processing data from sensors monitoring human movement or human activity. In particular, this document relates to tracking user performance across different activities using sensor data.

### SUMMARY

The following summary is intended to introduce the reader to various aspects of the detailed description, but not to define or delimit any invention.

A system, method, and computer program product for evaluating user performance across different activities is provided. In some examples, sensor data is obtained from sensors associated with a user while the user is performing one or more activities. The sensor data may be obtained from the user while the user performs activities corresponding to different activity categories, including gaming and non-gaming activities. The value of a standard athletic metric can be generated for the user for each activity using the corresponding sensor data. An activity category can also be determined for each activity. A user performance score for a given activity can be generated based on the corresponding standard athletic metric value and activity category.

A user performance score can be determined for each activity of a plurality of different activities performed by the user. Standard athletic metric values generated for the same standard athletic metric can be used to determine the corresponding user performance score for each activity. This can allow a user's performance to be tracked in a consistent manner while engaging in both gaming and non-gaming activities. The user performance scores can be used to generate a global user performance score for the user across multiple activities. The global user performance score can be used to obtain rewards or compare results against other users in an interactive activity environment, such as a gaming ecosystem for example. This may encourage users to engage in physical activity across multiple real-world and virtual environments by providing a consistent incentive structure for different types of activities.

According to some aspects, a method for evaluating user performance across different activities using sensor data from a plurality of sensors positioned to contact a user is provided, the method comprising: obtaining sensor data from the plurality of sensors, wherein the sensor data is collected while the user performs one or more activities, the one or more activities comprising at least one of a gaming activity or a non-gaming activity; for each activity of the one or more activities, generating a standard athletic metric value from the sensor data corresponding to that activity; determining an activity category of that activity; and generating a user performance score from the standard athletic metric value and the activity category.

Further examples of a method for evaluating user performance across different activities are shown and described herein.

According to some aspects, a system for evaluating user performance across different activities includes a plurality of sensors positionable to contact a user; one or more processors communicatively coupled to the plurality of sensors; wherein the one or more processors are configured to: obtain sensor data from the plurality of sensors, wherein the sensor data is collected while the user performs one or more activities, the one or more activities comprising at least one of a gaming activity or a non-gaming activity; for each activity of the one or more activities, generate a standard athletic metric value from the sensor data corresponding to that activity; determine an activity category of that activity; and generate a user performance score from the standard athletic metric value and the activity category.

Further examples of a system for evaluating user performance across different activities are shown and described herein.

According to some aspects, a non-transitory computer readable medium stores computer-executable instructions, which, when executed by a computer processor, cause the computer processor to carry out a method for evaluating user performance across different activities using sensor data from a plurality of sensors positioned to contact a user is provided, the method comprising: obtaining sensor data from the plurality of sensors, wherein the sensor data is collected while the user performs one or more activities, the one or more activities comprising at least one of a gaming activity or a non-gaming activity; for each activity of the one or more activities, generating a standard athletic metric value from the sensor data corresponding to that activity; determining an activity category of that activity; and generating a user performance score from the standard athletic metric value and the activity category.

The non-transitory computer readable medium can store computer-executable instructions, which, when executed by a computer processor, cause the computer processor to carry out the method for evaluating user performance across different activities, where the method is described herein.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a block diagram illustrating an example of a system for evaluating user performance across different activities;

FIG. 2 is a diagram illustrating an example of a wearable device incorporating a sensing unit that can be used in the system of FIG. 1;

FIG. 3 is a flowchart illustrating an example of a method for evaluating user performance across different activities; and

FIG. 4 is a screenshot illustrating an example graphical user interface that may be displayed to a user of the system shown in FIG. 1.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

The terms “coupled” or “coupling” as used herein can have several different meanings depending on the context in which these terms are used. For example, the terms coupled or coupling can have a mechanical, electrical or communicative connotation. For example, as used herein, the terms coupled or coupling can indicate that two elements or devices are directly connected to one another or connected to one another through one or more intermediate elements or devices via an electrical element, electrical signal, or a mechanical element depending on the particular context. Furthermore, the term “communicative coupling” may be used to indicate that an element or device can electrically, optically, or wirelessly send data to another element or device as well as receive data from another element or device.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof. Furthermore, the phrase “at least one of A and B” is intended to mean only A, only B, or a combination of A and B.

Terms of degree such as “substantially”, “about”, and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

The systems, methods, and devices described herein may be implemented as a combination of hardware or software. In some cases, the systems, methods, and devices described herein may be implemented, at least in part, by using one or more computer programs, executing on one or more programmable devices including at least one processing element and a data storage element (including volatile and non-volatile memory and/or storage elements). These devices may also have at least one input device (e.g. a pushbutton keyboard, mouse, a touchscreen, a foot-operated controller, and the like), and at least one output device (e.g. a display screen, a printer, a wireless radio, and the like) depending on the nature of the device.

Some elements that are used to implement at least part of the systems, methods, and devices described herein may be implemented via software that is written in a high-level procedural language such as object-oriented programming. Accordingly, the program code may be written in any suitable programming language such as Python or C, for example. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific, and predefined manner in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

The present disclosure relates in general to a system, method, and device that can be used to track and monitor user activity. The system, method, and device can evaluate user performance for one or more users across different activities.

Sensor data (and optionally other biometric data) can be obtained from a user. The sensor data can be obtained while the user performs one or more activities. The sensor data can be used to generate standard athletic metric values based on the user's performance in each activity. A user performance score can be generated for an activity based on the standard athletic metric value and the activity category of the activity. Multiple user performance scores can be generated for the user corresponding to multiple different activities, including activities in different activity categories, by calculating standard athletic metric values for the same standard athletic metric for each activity. A global user performance score can be determined based on the user performance scores generated for each individual activity.

The system, method and device described herein can use sensors positioned to contact the users to collect sensor data relating to the user's performance in one or more activities. For example, the sensor may be mounted to the user using one or more wearable devices. Users may participate in various different categories of activities while the sensors are mounted thereto. For example, users may participate in one or more non-gaming activities and/or one or more screen-based gaming activities, and/or one or more non-screen-based gaming activities.

The sensors can include force sensors. The force sensors can be positioned underfoot of the user. For example, force sensors can be provided in the insole of a shoe or within the footwear worn by the individual. The force data acquired by the force sensors can be used to determine the level of force applied by an individual's foot when performing actions such as walking, running, jumping or cycling for example during a particular activity. This force data can be used to derive additional force derivatives or force-based metrics, such as the force output, mean force, peak force, power and so forth for the individual.

Directly measuring the force (or pressure) applied by an individual using underfoot force sensors (as opposed to deriving the force data from other sensors such as accelerometers) can contribute to more accurate calculations of force-related metrics such as power. As used herein, the term “force” is used broadly and can refer to raw force (i.e. with units of N), or pressure resulting from a raw force (i.e. with units of N/m²).

The sensors can also include one or more inertial measurement units (IMUs). The IMUs can be positioned at various locations on the corresponding contributing user. IMU data from the one or more IMUs can be used to derive additional IMU-based metrics.

The sensor data from a user can be used to generate one or more standard athletic metric values for the user. The standard athletic metric value can be generated to provide a measurable indication of the intensity or effort exerted by the user while performing an activity. Various different athletic metrics, such as power and/or work/energy expenditure, can be defined as the standard athletic metric for which standard athletic metric values can be generated for a user. For example, the standard athletic metric may be defined as a level or rate of energy expenditure for the user while performing the activity. Values for the same standard athletic metric can be generated for the user while performing different activities and while performing activities associated with different activity categories. The standard athletic metric can be defined as a consistent metric for all activities. This can provide a way of comparing the athletic intensity or effort exerted by users across a range of activities.

Optionally, a set of force sensors can be associated with a corresponding IMU. The corresponding IMU can be configured to collect inertial measurement data relating to movement of the same foot under which the force sensors are positioned. This can facilitate the determination of standard athletic metrics based on a combination of force sensor data and IMU data. For instance, standard athletic metric values may be generated using a combination of force sensor data and IMU data.

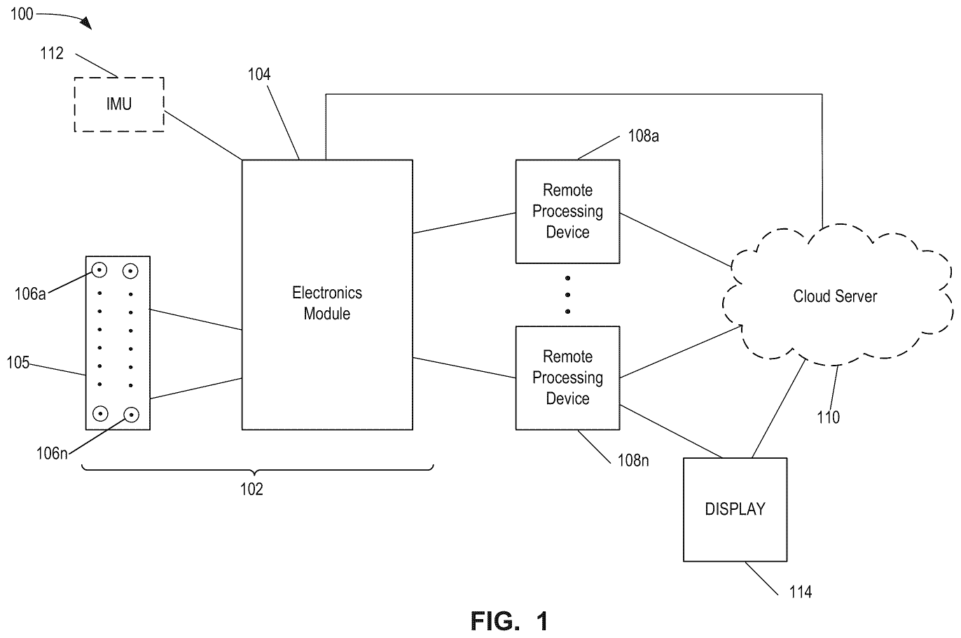

Referring now to FIG. 1, shown therein is a block diagram illustrating an example system 100 that can be used to evaluate user performance. System 100 includes an input unit 102 (also referred to herein as an input device), one or more processing devices 108 (also referred to herein as a receiving device or an output device), an optional remote cloud server 110 and a display 114.

The input unit 102 can be associated with a corresponding user. Each input unit 102 generally includes a corresponding sensor set. The input unit 102 can obtain sensor data from the associated user using the corresponding sensor set.

The set of sensors for each input unit 102 can include one or more sensors configured to obtain measurements of various parameters relating to human movement or human activity. For example, the set of sensors can include force sensors operable to measure a force applied by the corresponding user.

Alternatively or in addition, the set of sensors can include an inertial measurement unit operable to measure various parameters relating to movement by the corresponding user.

Alternatively or in addition, various other types of sensors may be included in the sensor set of an input unit 102, such as, for example, a shear sensor, a weight sensor, a body-mass-index sensor, a temperature sensor, a moisture sensor, a heart rate sensor, a heart rate variability sensor, a blood pressure sensor, a blood flow sensor, a cardiac output sensor, a perfusion sensor, an inductance sensor, an odor sensor, a taste sensor, a hydration sensor, a respiratory flow rate sensor, a limb positioning measurement device, a chemical marker sensor, a blood glucose sensor, a sweat sensor, a blood alcohol sensor, an oxygen sensor, a carbon dioxide sensor, a drug level sensor, an electrolyte sensor, a pH sensor, an acidity sensor, an EEG sensor, an EMG sensor, an ECG sensor, a lung function meter, an impairment sensor, a sleep sensor, a body fat sensor, a height sensor, a fatigue sensor, a facial recognition sensor, a voice sensor, an emotion sensor, a stress sensor, an endorphin sensor, a cortisol sensor, an adrenaline sensor, an infrared sensor, a bacterial load sensor, a motion capture sensor, a timer, a weather sensor, a GPD sensor, an audio sensor, a light sensor, an altimeter, radar, lidar, a milestone sensor, an equipment-based sensor, a proximity sensor, and so on.

In the example illustrated, input unit 102 includes a force sensing unit 105 containing a plurality of force sensors 106a-106n and an optional inertial measurement unit 112.

Optionally, the sensor set included in an input unit 102 may include multiple force sensing units 105 and/or inertial measurement units 112 for the same user (e.g. to obtain sensor data from different locations on the user). For example, the sensor set included in an input unit 102 may include a separate sensing unit 105 (and optionally a separate IMU 112) for each foot of an individual. The sensor set may include a first force sensing unit 105 associated with a first foot of the user and a second force sensing unit 105 associated with the second foot of the user.

Alternatively, a single sensing unit 105 may be used to acquire force sensor data for both feet of an individual. This may be the case where the sensing unit 105 is incorporated into fitness equipment such as bike pedals. In such cases, the force sensor data acquired by the sensing unit 105 may be associated with individual feet through further processing by electronics module 104 and/or processing device 108.

The sensor set included in an input unit 102 may also include one or more inertial measurement units 112 mounted at respective locations on the user.

Optionally, the sensor set included in the input unit 102 can include an inertial measurement unit 112 associated with each force sensing unit. This may allow IMU sensor data and force sensor data to be obtained from the same location on the user's body. IMU data acquired by the IMU 112 associated with each foot may be used to associate the force sensor data acquired by a single sensing unit 105 with the corresponding foot.

Optionally, the IMU 112 can also be positioned underneath an individual's foot. This may be the case, for instance, where IMU 112 and force sensing unit 105 are associated and provided as a combined sensor unit. However, the IMU 112 need not be positioned underfoot so long as the IMU 112 can collect inertial measurement data relating to the user's movement or motion as required by the particular application.

Alternatively or in addition, the sensor set included in the input unit 102 can include one or more inertial measurement units 112 at locations where no force sensors are present. This may allow IMU sensor data to be obtained relating to movement by a user at locations where forces may not be applied (or applied directly in a manner that would be detectable to a force sensor).

Optionally, an input unit 102 may include multiple electronic modules 104. Each electronic module 104 may be associated with a different subset of sensors within the corresponding sensor set included in the input unit 102.

The plurality of force sensors 106a-106n in a force sensing unit 105 can be configured to collect force sensor data from a user. For example, the plurality of force sensors 106 can be positioned underfoot of an individual performing an activity or other type of movement.

An IMU 112 can include one or more sensors for measuring the position and/or motion of the wearable device. For example, IMU 112 may include sensors such as one or more of a gyroscope, accelerometer (e.g., a three-axis accelerometer), magnetometer, orientation sensor (for measuring orientation and/or changes in orientation), angular velocity sensor, and inclination sensor. Generally, IMU 112 includes at least an accelerometer. The IMU 112 also typically includes a gyroscope.

The sensors may be provided using a wearable device and/or fitness equipment. As will be described in further detail below, each input unit 102 may for example be provided using (e.g. combined with, or integrated into) one or more carrier units such as one or more wearable devices and/or pieces of fitness equipment.

Preferably, the carrier unit is a portable carrier unit such as a wearable device or portable fitness equipment such as a bike seat or bike pedals for instance. Providing the plurality of sensors using one or more portable carrier units can ensure that the same sensors are used to obtain the sensor data for a wide range of activities, including activities at different locations as well as both indoor and outdoor activities.

The carrier unit can be configured to position the sensors (e.g. force sensors 106 and/or IMU 112) in contact with (or in close proximity to) an individual's body to allow the sensors to measure an aspect of the activity being performed by the individual. The plurality of sensors may be configured to measure a particular sensed variable at a location of an individual's body when the carrier unit is engaged with the individual's body (e.g. when the individual is wearing a wearable device containing the sensors).

In some examples, a carrier unit may include one or more wearable devices. The wearable devices can be manufactured of various materials such as fabric, cloth, polymer, or foam materials suitable for being worn close to, or in contact with, a user's skin. All or a portion of the wearable device may be made of breathable materials to increase comfort while a user is performing an activity.

In some examples, the wearable device may be formed into a garment or form of apparel such as a band, a watch, an arm mount, an ankle mount (e.g. a Stryd pod), headwear, a shirt, shorts, a sock, a shoe, a sleeve, and a glove (e.g. a tactile glove). Some wearable devices such as socks or sleeves may be in direct contact with a user's skin. Some wearable devices, such as shoes, may not be in direct contact with a user's skin but still positioned within sufficient proximity to a user's body to allow the sensors to acquire the desired readings.

In some cases, the wearable device may be a compression-fit garment. The compression-fit garment may be manufactured from a material that is compressive. A compression-fit garment may minimize the impact from “motion artifacts” by reducing the relative movement of the wearable device with respect to a target location on the individual's body. In some cases, the wearable device may also include anti-slip components on the skin-facing surface. For example, a silicone grip may be provided on the skin-facing surface of the wearable device to further reduce the potential for motion artifacts.

In some examples, the wearable device can be worn on a foot. For example, the wearable device may be a shoe, a sock, or an insole, or a portion of a shoe, a sock, or an insole. The wearable device may include a deformable material, such as foam. This may be particularly useful where the wearable device is a shoe or insole.

The plurality of sensors can be positioned to acquire sensor readings from specified locations on an individual's body (via the arrangement of the sensors on the carrier unit). The sensors can be integrated into the material of the carrier unit (e.g. integrated into a wearable device or fitness equipment). Alternatively, the sensors can be affixed or attached to the carrier unit, e.g. printed, glued, laminated or ironed onto a surface, or between layers, of a wearable device or fitness equipment.

In system 100, the plurality of force sensors 106a-106n can be arranged to measure force underneath the foot (underfoot) of an individual. For clarity, the below description relates to a carrier unit in the form of an insole. The insole carrier unit may be provided in various forms, such as an insert for footwear, or integrated into a shoe. However, other carrier units may be implemented using the systems and methods described herein, such as the wearable devices described above.

Incorporating the sensor set into a carrier unit in the form of a wearable device may be desirable as it allows sensor data to be obtained from a user at various locations and without requiring specifically configured fitness or gaming equipment. This may allow user activity to be monitored while participating in different categories of activities including gaming and non-gaming activities. In addition, this may allow user activity to be monitored while participating in activities at many different locations (e.g. including indoor and outdoor locations) including non-gaming activities as well as gaming activities at fixed locations and/or mobile gaming activities.

The below description relates to an insole in which the plurality of sensors 106 are force sensors. Various types of force sensors may be used, such as force sensing resistors (also referred to as sensing elements), pressure sensors, piezoelectric tactile sensors, elasto-resistive sensors, capacitive sensors or more generally any type of force sensor that can be integrated into a wearable device or fitness equipment capable of collecting force data underfoot.

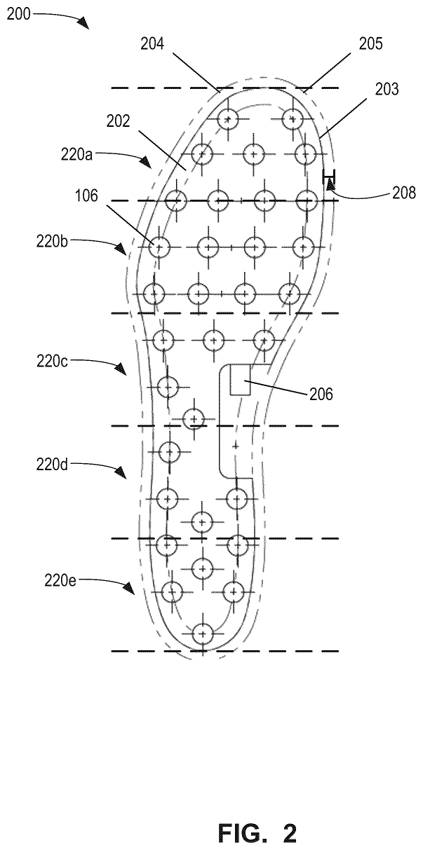

The plurality of force sensors 106 may be arranged into a sensor array. As used herein, the term sensor array refers to a series of sensors arranged in a defined grid. The plurality of force sensors 106 can be arranged in various types of sensor arrays. For example, the plurality of force sensors 106 can be provided as a set of discrete sensors (see e.g. FIG. 2). A discrete sensor is an individual sensor that acquires a sensor reading at a single location. A set of discrete sensors generally refers to multiple discrete sensors that are arranged in a spaced apart relationship in a sensing unit.

Sensors 106a-106n may be arranged in a sparse array of discrete sensors that includes void locations where no sensors 106 are located. Alternatively, sensors 106a-106n may be arranged in a continuous or dense sensor array in which sensors 106 are arranged in a continuous, or substantially continuous manner, across the grid.

Discrete sensors can provide an inexpensive alternative to dense sensor arrays for many applications. However, because no sensors are positioned in the interstitial locations between the discrete sensors and the void locations external to the set of discrete sensors, no actual sensors readings can be acquired for these locations. Accordingly, depending on the desired resolution for the force sensor data, sensor readings may be estimated (rather than measured) at the interstitial locations and at the void locations external to the set of discrete sensors in order to provide sensor data with similar resolution to a dense sensor array. Alternatively, where lower resolution force sensor data is sufficient, sensor readings may not necessarily be estimated.

Various interpolation and extrapolation techniques may be used to estimate sensor values at interstitial locations and external void locations. In some cases, sensor values may be estimated using the methods for synthesizing sensor data described in Applicant's co-pending patent application Ser. No. 17/988,468 filed on Nov. 16, 2022 entitled “SYSTEM AND METHOD FOR SYNTHESIZING SENSOR READINGS”, the entirety of which is incorporated herein by reference. In some cases, sensor values may be estimated using the methods for synthesizing sensor data described in Applicant's co-pending patent application Ser. No. 18/183,642 filed on Mar. 14, 2023 entitled “SYSTEM AND METHOD FOR DETERMINING USER-SPECIFIC ESTIMATION WEIGHTS FOR SYNTHESIZING SENSOR READINGS”, the entirety of which is incorporated herein by reference.

System 100 can be configured to implement a method of evaluating user performance across different activities. The method of evaluating user performance may be implemented using a controller of the input device 102, a remote processing device 108, and/or cloud server 110.

As shown in FIG. 1, input unit 102 includes an electronics module 104 coupled to the plurality of sensors 106 and to IMU 112. In some cases, the electronics module 104 can include a power supply, a controller, a memory, a signal acquisition unit operatively coupled to the controller and to the plurality of sensors 106 (and to IMU 112), and a wireless communication module operatively coupled to the controller.

Generally, the sensing unit refers to the plurality of sensors and the signal acquisition unit. The signal acquisition unit may provide initial analog processing of signals acquired using the sensors, such as amplification. The signal acquisition unit may also include an analog-to-digital converter to convert the acquired signals from the continuous time domain to a discrete time domain. The analog-to-digital converter may then provide the digitized data to the controller for further analysis or for communication to a remote processing device 108 or remote cloud server 110 for further analysis.

Optionally, the electronics module 104 may include a controller or other processing device configured to perform the signal processing and analysis. In such cases, the controller on the electronics module may be configured to process the received sensor readings (e.g. to generate a standard athletic metric value). In some cases, the controller may be coupled to the communication module (and thereby the sensing unit) using a wired connection such as Universal Serial Bus (USB) or other port.

The electronics module 104 can be communicatively coupled to one or more remote processing devices 108a-108n, (e.g. using a wireless communication module such as Bluetooth, Bluetooth Low-Energy, WiFi, ANT+ IEEE 802.11, etc.). The remote processing devices 108 can be any type of processing device such as (but not limited to) a personal computer, a tablet, a gaming system, and a mobile device such as a smartphone, a smartwatch or a wristband. The electronics modules 104 can also be communicatively coupled to remote cloud server 110 over, for example, a wide area network such as the Internet.

Each remote processing device 108 and optional remote cloud server 110 typically includes a processing unit, an output device (such as a display, speaker, and/or tactile feedback device), a user interface, an interface unit for communicating with other devices, Input/Output (I/O) hardware, a wireless unit (e.g. a radio that communicates using CDMA, GSM, GPRS or Bluetooth protocol according to standards such as IEEE 802.11a, 802.11b, 802.11g, or 802.11n), a power unit, and a memory unit. The memory unit can include RAM, ROM, one or more hard drives, one or more flash drives or some other suitable data storage elements such as disk drives, etc.

The processing unit controls the operation of the remote processing device 108 or the remote cloud server 110 and can be any suitable processor, controller, or digital signal processor that can provide sufficient processing power depending on the desired configuration, purposes, and requirements of the system 100.

The display 114 can be any suitable display that provides visual information. For instance, the display 114 can be a cathode ray tube, or a flat-screen monitor and the like if the remote processing device 108 or remote cloud server 110 is a desktop computer. In other cases, the display 114 can be a display suitable for a laptop, tablet or handheld device, such as an LCD-based or LED-based display and the like. In still other cases, the display 114 can be a display suitable for a virtual-reality or augmented reality system, such as a virtual reality headset or smart glasses for example. Although only one display 114 is shown, it should be understood that there may be multiple displays in system 100. For example, each user may have an associated display 114.

Additionally, although the example system 100 shown in FIG. 1 illustrates a single input unit 102 corresponding to a single user it should be understood that multiple users may interact with system 100, each having their own associated input unit 102 and optionally one or more corresponding remote processing devices 108.

System 100 can generally be used to monitor and evaluate user performance based on sensor readings received from a plurality of sensors positioned to contact a user. The sensor readings can be used to generate standard athletic metric values and user performances scores corresponding to the user's activity while participating in one or more activities. The sensor readings, standard athletic metric values, performance scores and other derived data may be monitored, stored, and analyzed for the user and optionally for a plurality of additional users.

The sensor readings can be used to determine biometric data such as standard athletic metric values for one or more activities. The activities are typically movement-based activities, such as non-gaming athletic activities and movement-based gaming activities.

The user performance scores generated for individual activities can be used to determine a global user performance score that can be accumulated based on the user's participation in various different activities and activity categories. This can provide a standardized way to incentivize or reward users for movement in both non-gaming and gaming environments.

Standard athletic metrics are generally defined to include any measure of athletic intensity/effort of a user that can be directly measured or calculated using the sensor data obtained from the plurality of sensors positioned to contact the user. The generation of standard athletic metric values may also encourage movement while users are performing gaming activities. For example, the level of intensity/effort reflected in the standard athletic metric value may be used to increase/decrease the user performance scores generated for the user in order to encourage the user to increase their level of movement.

System 100 can be used to track, monitor and evaluate user performance across different categories of movement-based activities. User performance scores can be calculated across all of the different categories of movement-based activities. Global user performance scores can be accumulated across all of the different categories of movement-based activities.

In general, movement-based activities relate to activities in which a user performs some form of physical movement to engage in the activity. This can include both gaming and non-gaming movement activities. Gaming movement activities generally refer to games in which at least some of the user's interaction with the game involves the user's movements being tracked or monitored in some manner.

The activity categories can include a non-gaming movement activity category. The non-gaming movement activity category can generally include any movement activity that a user performs that is unrelated to a game. This can include any non-gaming movement activity that the user performs in any environment or location (including indoor or outdoor environments). For example, going for a run, playing a basketball game, attending a yoga class, lifting a sofa, etc. are all examples of non-gaming movement activities.

The activity categories can include a non-screen-based gaming activity category. The non-screen-based gaming activity category can generally include any movement-basedgame for which a screen is not required during gameplay. For example, a non-screen-based game may require a user to go for a run around their block to outrun a monster. The user does not need to look at a screen while playing the game but may review their progress in the game on a screen post-activity, or even intermittently while the activity is ongoing.

The activity categories can include a screen-based gaming activity category. The screen-based gaming activity category can generally include any movement-based game that requires a screen during gameplay. For example, a dancing game may require users to perform a sequence of steps displayed on a screen. Another screen-based game may require a user to walk around their city and locate virtual treasures using a mobile device (e.g. through an artificial reality overlay displayed on the screen of their mobile device).

Sensor data can be collected using the sensors provided by input devices 102. Optionally, the sensor data can be provided to a mobile application operating on a remote processing device 108. The remote processing device 108 can be configured to generate the standard athletic metric values and/or user performance scores using the mobile application.

Alternatively or in addition, some or all of the standard athletic metric values and/or user performance scores may be generated by electronic module 104. The generated data can then be provided to the mobile application on the remote processing device 108.

The mobile application can also allow a user to see and review various data relating to the user's performance in one or more activities, including, for example, standard athletic metric values and user performance scores associated with specific activities as well as global user performance scores and other related data. For example, a user may access the mobile application to wirelessly couple the remote processing device 108 to electronic module 104, record data, review past data, engage in gaming activities and various other actions (e.g. calibrating the input device 102). Optionally, other wearable sensors or health applications can be used to provide data for the system (e.g. sleep tracking, nutritional tracking, etc.).

Aspects of the monitoring, storage and analysis of sensor data, standard athletic metric values, user performance scores, and other scores or metrics may be performed by one or more of the input unit 102, and/or a remote processing device 108, and/or the cloud server 110. For example, a non-transitory storage memory of one or more of the input unit 102, and/or a remote processing device 108, and/or the cloud server 110 can store various user related data such as session logs containing sensor data from a user, historical activity data for a user including historical metrics and user performance scores, a global user performance score and other data derived or generated therefrom.

A remote cloud server 110 may provide additional processing resources not available on the input unit 102 or the remote processing device 108. For example, some aspects of processing the sensor readings acquired by the sensors 106 may be delegated to the cloud server 110 to conserve power resources on the input unit 102 or remote processing device 108. The cloud server 110, input unit 102 and remote processing device 108 may communicate in real-time to generate standard athletic metric values and user performance scores and other related data.

System 100 can also provide an interactive gaming ecosystem that includes a plurality of users. The gaming ecosystem can allow users to interact with other users and to compare user data such as metrics and scores, and compete against other users in various gaming and non-gaming activities. The gaming ecosystem provided by system 100 may allow users to compete against one another in competitions such as tournaments.

Referring now to FIG. 2, shown therein is an example of an insole 200 that includes a sensing unit 202. The insole 200 is an example of a sensing unit that may be included in an input device 102 in the system 100 shown in FIG. 1. The insole 200 may be the footwear insert described in PCT Application No. PCT/CA2020/051520, the entirety of which is incorporated herein by reference.

The insole 200 includes a sensor unit 202 and an optional liner 204. The liner 204 can provide a protective surface between the sensor unit 202 and an individual's foot. The liner 204 may have a slightly larger profile as compared to the sensor unit 202. That is, the outer perimeter 203 of the sensor unit 202 may be inwardly spaced from the outer perimeter 205 of the liner 204 by an offset 208. The offset 208 may be substantially consistent throughout the perimeter of the sensor unit 202 such that the sensor unit 202 is completely covered by the liner 204.

Optionally, the sensor unit 202 can include an IMU (not shown). The sensor unit 202 can also include a connector 206. The connector 206 may provide a coupling interface between the plurality of force sensors 106 (and the optional inertial measurement unit) and an electronics module (not shown) such as electronics module 104. The coupling interface can allow signals from the force sensors 106 and/or IMU to be transmitted to the electronics module. In some cases, the coupling interface may also provide control or sampling signals from the electronics module to the force sensors 106 and/or IMU.

The arrangement of force sensors 106 in the sensor unit 202 is an example of a sparse sensor array that may be used to collect force sensor data. In alternative examples, various different types of force sensors, force sensor arrays, and arrangements of force sensors may be used. For example, sensor units containing a dense force sensor array (e.g. a Pedar® insole or Tekscan® system) may also be used.

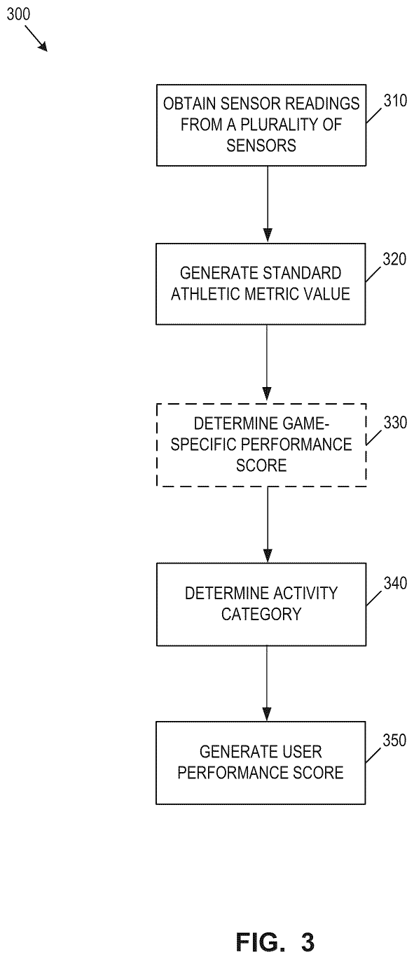

Referring now to FIG. 3, shown therein is an example method 300 for evaluating user performance across different activities. The method 300 may be used with a plurality of sensors configured to measure human movement or human activity, such as force sensors 106 and/or IMU 112. Method 300 is an example of a method for evaluating user performance in which sensor readings acquired from a user are used to generate standard athletic metric values and user performance scores relating to the user participating in one or more activities.

At 310, sensor data can be obtained from a plurality of sensors. The plurality of sensors can be positioned to contact a user while the user performs one or more activities.

The sensors can be positioned at specified locations on a carrier unit. The sensors can be configured to measure data relating to human movement or activity. A plurality of sensor readings can be obtained from the sensors associated with the user.

For example, the sensor set can include a plurality of force sensors positioned to obtain force sensor data from a user while the user is performing an activity. The force sensors can be provided using one or more corresponding carrier units. The sensor data for that user can thus include the force sensor data from the plurality of force sensors.

The plurality of force sensors may be positioned underfoot (i.e. underneath the foot) of a user performing a physical activity. The force sensors can thus measure the force applied by the user's foot while performing the physical activity.

As shown in FIG. 2, the plurality of sensors may be force sensors provided at various locations of an insole. The force sensors can measure force applied to the insole during movement activities, such as walking, running, jumping, or cycling for example.

The sensor set may also include one or more IMUs. The sensor data acquired at 310 can include IMU sensor data received from the one or more IMUs.

The sensor data acquired at 310 may be acquired as a time-continuous set of sensor readings. This may provide a time-continuous set of sensor data that can be used to generate standard athletic metric values as time-continuous values and/or average values. Depending on the nature of the sensors and the signal preprocessing performed, the time-continuous sensor data may be discretized, e.g. using an analog to digital conversion process. Even where the sensor data is discretized, the set of sensor data may allow the standard athletic metric values to be generated as (discretized) time-continuous values and/or average values.

The sensor data may be obtained while the user participates in one or more activities. The sensor data can include activity-specific sensor datasets that include discrete portions of sensor data obtained when the user was performing a specific activity.

The sensor data can be obtained while a user participates in a plurality of activities. Accordingly, the sensor data can include a plurality of activity-specific sensor datasets. Each activity-specific sensor datasets can include the sensor data obtained while the user was performing a corresponding activity from the plurality of activities. Steps 320-350 may be repeated for each activity a user performs. Furthermore, as should be apparent, steps 320-340 may be performed in a different order and some or all of steps 320-340 may be performed concurrently.

At 320, a standard athletic metric value can be generated from the sensor data obtained at 310. The standard athletic metric value can be generated for each activity performed by a user. The standard athletic metric value for a given activity can be generated using the sensor data obtained at 310 for that user while performing the given activity.

A standard athletic metric generally refers to a predefined and fixed metric that is representative of the intensity or effort exerted by a user when they perform an activity. A standard athletic metric may be defined as a biometric that can be calculated directly from the sensor data obtained at 310.

The standard athletic metric can be selected as a metric that can be calculated for any movement-based activity. This can allow the same standard athletic metric to be calculated and compared across multiple activities. Using a fixed standard athletic metric for each and every activity can allow users to compare intensity or effort levels against other users who are participating in the same or different activities. This may encourage users to engage in movement-based activities in a competitive manner with other users who might have different interests in terms of the specific gaming or non-gaming activities they like to participate in.

Various different types of standard athletic metrics may be generated. For example, a standard athletic metric may be defined as a user's energy expenditure or work (e.g. calories burned performing the activity). Alternatively or in addition, a standard athletic metric may be defined as a user's power output (e.g. rate of energy expenditure, in Watts), or a user's rate of power output (e.g. in Watts/hour).

The standard athletic metrics can also be calculated as different metric types. For example, a standard athletic metric can be calculated as an instantaneous value (e.g. rate of energy expenditure at one point in time during a run) and/or it can be an accumulated value (e.g. total energy expenditure over the course of a run).

Users may also review their metrics during or after an activity (e.g. through a mobile application on a remote processing device 108 such as a smartphone or smartwatch). This real-time feedback may encourage users to continue to engage with the activity at a higher level of intensity or increase their intensity.

Optionally at 330 a game-specific performance score can be determined. A game-specific performance score can be calculated only for gaming activities. The game-specific performance score can be calculated to reflect the user's ability to fulfill the objectives of the particular gaming activity they are participating in.

The calculation of the game-specific performance score will vary depending on the specific gaming activity being performed. The game-specific performance score can be calculated based on the sensor data obtained at 310 and the objectives associated with the specific gaming activity. For example, the sensor data may be used to evaluate whether a user has completed a gesture correctly within a specified time window or with the correct amount of force in the right area of the foot.

The calculation of the game-specific performance score can also include modification factors such as multipliers and bonuses for successful completion of objectives including streaks, skillful movement combinations, and/or other unique game experiences such that performing the same in-game action may not yield the same game-specific performance score each time.

Users may review their game-specific performance score during or after an activity (e.g. through a mobile application on a remote processing device 108 such as a smartphone or smartwatch).

The game-specific performance scores can be stored using a non-transitory storage memory, such as may be provided by remote processing device 108 and/or server 110. The game-specific performance scores for a particular session can each be stored to allow user statistics to be calculated and to allow historical game-specific performance scores to be reviewed and compared. Game-specific performance scores may also be displayed over specified windows (e.g. daily totals, weekly totals, or seasonal statistics for example).

At 340, an activity category can be determined for a given activity that the user participates in. As noted above, the sensor data may be obtained while the user participates in one or more activities. An activity category can be determined for each activity in which the user participates.

The activity category can include one or more gaming activity categories and a non-gaming activity category. The gaming activity categories can include a screen-based gaming activity category and a non-screen-based gaming activity category.

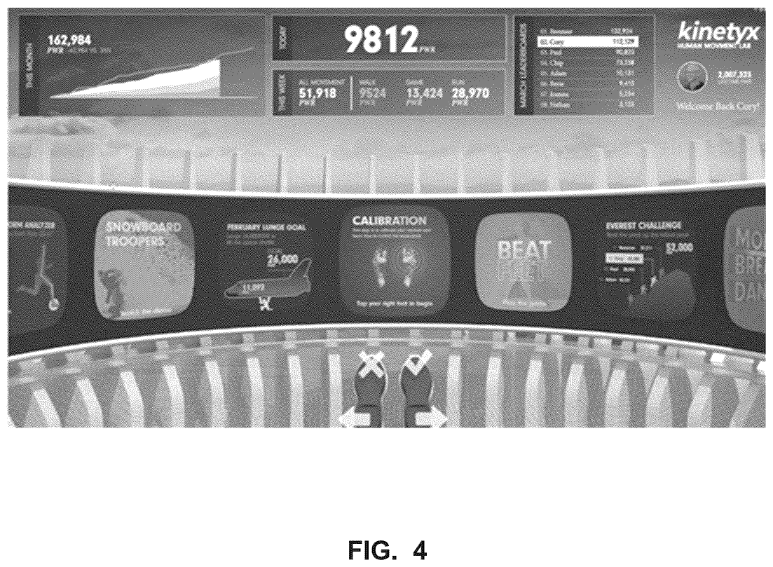

The activity category can be determined in various ways. For example, a user may select a particular game by interacting with a remote processing device 108 (e.g. by selecting a gaming activity through a mobile application). The activity category can then be determined based on the game selected by the user (e.g. a screen-based gaming activity category or a non-screen-based gaming activity category). For example, if a user selects the “Snowboard Troopers” game shown in the user interface of FIG. 4, the activity category can be identified as a screen-based gaming activity. Sensor data collected when a user has not selected a gaming activity may be automatically identified as a non-gaming activity.

Optionally, a specific activity type may be determined for a given activity. The specific activity type may be used in generating the standard athletic metric value for the activity. For instance, determining the specific activity type may allow the user's level of energy expenditure to be determined more accurately when generating the standard athletic metric value from the sensor data.

Optionally, the specific activity type may be identified by a user providing input indicating the specific type of activity being performed (e.g. running, playing badminton, dancing etc.). Alternatively, an activity classification method can be applied to the sensor data to determine the corresponding activity type being performed (e.g. running, cycling, etc.).

For example, an activity type machine learning model (e.g. a neural network) can be trained to distinguish between different activities based on IMU data received from one or more IMUs 112 and/or force sensor data received from one or more force sensing units 105. Optionally, the activity type machine learning model can be trained to determine the activity type based on sensor data solely from sensors (e.g. IMUs and/or force sensors) positioned underfoot of a user.

An example of an activity classification method that may be used to classify the sensor data is described in U.S. Pat. No. 11,526,749 entitled “METHOD AND SYSTEM FOR ACTIVITY CLASSIFICATION”, the entirety of which is incorporated herein by reference.

At 350, a user performance score can be generated using the standard athletic metric value (from 320) and the activity category (from 340). Generation of the user performance score may also rely on the game-specific performance score where relevant.

The user performance score for a given activity can be generated based on accumulated values of the standard athletic metric and game-specific performance score determined at 320 and 330 respectively. The activity category from 340 can be used to determine the function applied to the standard athletic metric value and optionally the game-specific performance score to generate the user performance score.

Determining a user performance score for an activity involves the accumulated value of the standard athletic metric for the user from the activity (e.g. total energy expended in Joules or calories) in addition to the game-specific performance score the user receives from playing the game (if they were indeed participating in a gaming activity rather than a non-gaming activity such as running).

If a user participates in a non-gaming activity (as identified at 340), the standard athletic metric value from 320 may be the only contributor to the user performance score. For example, the user performance score for a non-gaming activity may be determined as shown in Equation 1:

userperformancescore_{non-game}=n₁*SAM (1)

where n is a normalization factor and SAM is the standard athletic metric value.

If a user participates in a gaming activity (as identified at 340) the standard athletic metric value from 320 and the game-specific performance score from 330 may both be used to calculate the user performance score. For example, the user performance score may be calculated as a weighted sum of the standard athletic metric value and the game-specific performance score as shown in Equation 2:

userperformancescore_{game}=W₁*n₁*SAM+W₂*n₂* gameperfscore (2)

where n is a normalization factor, W is a weight value, SAM is the standard athletic metric value, and gameperfscore is the game-specific performance score.

The weight values W can be defined to specify the level of contribution that the standard athletic metric value and game-specific performance score have on the user performance score. The weight values can be defined and adjusted as desired by an operator or administrator of system 100.

Optionally, the weight values can be defined such that the standard athletic metric value contributes more to the user performance score than the game-specific performance score. This can ensure that the user performance score is more closely related to the user's level of intensity/exertion when performing the activity. This may also facilitate the comparison of user performance scores across different activities. Accordingly, W₁ can be defined to be a greater value than W₂ in Equation 2.

The normalization factors noted above are optional. However, normalization factors may be particularly desirable in competitive implementations (e.g. tournaments) to allow users to compete on equitable grounds.

The normalization factors may be defined to allow user performance scores to be determined fairly for different users. Normalization factors may be applied to user performance scores to account for factors such as mass/weight, age, gender, natural athletic ability, game skill (in the case of game-specific performance scores), and/or other physical characteristics.

For example, standard athletic metric values calculated from a plurality of force sensors positioned underfoot of users will result in certain users having larger standard athletic metric values than others since they naturally apply more force to the ground. For example, heavier users will naturally apply more force to the ground during running than lighter users. This will result in the generation of large standard athletic metric values for heavier users. However, despite the difference in standard athletic metric values between heavy and light users, the normalization factors allow users of different sizes to obtain the same user performance scores for performing equivalent activities.

For example, consider two individuals competing in a dancing game. User A is 50 kg and user B is 80 kg. If normalization factors are not included in the calculation of the user performance scores, user B would have a higher user performance score than user A due to user B's larger standard athletic metric value, even though both users compete at the same skill level. Accordingly, a normalization factor can be applied to user A's user performance score calculation (or to user B's user performance score calculation or to both) so that both users obtain the same user performance score for the same movements.

The normalization factors may also allow users to compare results against other users with different demographics or skill levels. For example, normalization factors may allow parents, children and grandparents with different skill levels and physical capabilities to achieve similar results when performing an activity. This can encourage a broad range of users to participate in movement-based activities.

Optionally, additional factors may be used to generate user performance scores. For example, certain activity categories (e.g. non-gaming activities) may be weighed more heavily than others. For example, if non-gaming activities are weighed more heavily than gaming activities, this may encourage users to increase the amount of non-gaming activities they perform. Alternatively or in addition, specific activities may be weighed more heavily to encourage greater physical activity (e.g., the game-specific performance score from a dancing game might contribute more towards the user performance score than the game-specific performance score from a driving game). Other examples of additional factors include a user fitness level, a user tournament experience level, a user's current global user performance score and so forth.

Equation 3 illustrates an example function for calculating a user performance score that incorporates additional factors:

userperformancescore_{game}=W₁*n₁*SAM+W₂*n₂*gameperfscore+W₃*n₃*factor₃+W₄*n₄*factor₄+ . . . (3)

where each factor represents an additional factor, and any number of additional factors can be added to the calculation.

However, it should be understood that Equation 3 is just an example, and additional factors could be incorporated into the calculation in any number of ways (e.g. subtracted from the calculation, applied as a multiplier to the other terms, etc.).

Optionally, modification factors such as welcome bonuses, personal milestones, group milestones, and so forth may be used to adjust user performance scores.

Optionally, the game-specific performance score (at 330) and/or user performance score (at 350) can be calculated using additional user data. The additional user data may include user health data such as sleep data, nutrition data, or other health-related data. For example, the user health data may be received from third-party applications and/or devices which track user health information such as sleep (e.g. Oura™), nutrition (e.g. MyFitnessPal™), or other health-related information.

The additional user data may also include user feedback data provided by the user. For example, users may complete pre-workout and post-workout surveys reporting information such as injuries, soreness, and rate of perceived exertion (RPE).

User performance scores may also be modified based on user interactions with the mobile application or system 100. The user performance score modifications can be defined to encourage users to interact with the system 100 in a desired manner. For example, user performance scores may be boosted or rewarded in response to additional data provided by the user. Alternatively or in addition, user performance scores may be boosted in response to user's performing sensor calibration (which also increases the accuracy of data collection).

User performance scores for specific activities can also be used to determine a global user performance score for a user. The global user performance score for a user may be determined based on the user performance scores generated for that user across multiple different activities. The global user performance score may be defined as an accumulated score value that is based on user performance scores over time. For example, a user's global user performance score may be calculated as a sum of all the user performance scores generated for that user over time.

Optionally, modification factors such as welcome bonuses, personal milestones, group milestones, and so forth may be used to adjust global user performance scores.

Global user performance scores may also be modified based on user interactions with the mobile application or system 100. The global user performance score modifications can be defined to encourage users to interact with the system 100 in a desired manner. For example, global user performance scores may be boosted or rewarded in response to additional data provided by the user. Alternatively or in addition, global user performance scores may be boosted in response to user's performing sensor calibration (which also increases the accuracy of data collection).

The user performance scores can also be used to determine other gaming-related metrics for the user. For example, a user can be associated with one or more user levels. The user levels generally refer to the experience and/or power of the user with a game or the system 100 as a whole. User levels may be used to compare users to one another, or to establish progression in fitness and experience over time.

Various different types of user levels may be determined for a given user. For example, an overall experience level may be determined for a user. The overall experience level can be defined to represent the user's experience with the system 100 over all the gaming and non-gaming activities the user has performance. The overall experience level of a user may be determined based on the user's global user performance score.

For example, a user's overall experience level may start at a base level and increase with continued play. One or more of the standard athletic metric values, game-specific performance scores, user performance scores, and global performance scores may be used to determine the progression towards a subsequent level. The standard athletic metric values and user performance scores may be achieved from any type of activity including a screen-based game, a non-screen-based game, or a non-game activity.

The experience required to reach a subsequent experience level may be based on a sliding scale, where a lower level is easier to achieve than a higher level. The sliding scale may make higher levels more difficult to achieve. For example, for a consistent sliding scale, a 1.25 multiplier for experience may be applied to every subsequent level, meaning level 3 requires 1.25× more experience than the experience required to reach level 2.

The system operator or administrator may also choose to alter level-up requirements to make the entire player population level distribution reflect a normal distribution. A normal distribution may improve user engagement.

Optionally, a user's overall experience level may only move to higher levels. Optionally, a user's overall experience level may move only one level at a time. For example, a user continues playing games and gaining experience. Practice increases the user's experience level from level 2 to level 3. Once level 3 is achieved the user will not return to level 2, but will instead advance to level 4. Unique conditions or performances may accelerate a user's progress towards the next goal.

A user may also have an associated tournament experience level. A user's tournament experience level can also be determined based on the standard athletic metric values, game-specific performance scores, and user performance scores. The tournament experience level may be used to match players appropriately for competitive activities such as tournaments or challenges.

System 100 may provide tournaments as an optional series of activities that users can enter to compete against other users. Tournaments can be created for users playing the same game/activity and/or they may be created for users playing different games/activities.

In both the same-game and different-game tournaments, users can be ranked based on one or more of standard athletic metric values, game-specific performance scores, and user performance scores earned during the tournament period. Every user that enters the tournament can start on an even playing field (e.g. a user performance score of zero at the start of the tournament) although handicaps may be included (e.g. to allow users with different capabilities to compete more equitably).

Optionally, a tournament may have player capacities, which limit the number of users that are able to enter the tournament.

Optionally, tournaments may include player-to-player competition, where each player competes by themselves against other individual players and/or may include team-to-team competition, where a group of players compete as a team, against other teams. For team-to-team competition, users may have the option to form their own team or may be randomly placed in teams for the tournament duration. The end of the tournament may be designated by the number of games played, user performance scores, amount of time played, or a set end time.

When a tournament ends, users can be ranked based on their performance during the tournament. The final ranking may be used to generate rewards for higher ranking players. For example, the top three ranked players in a tournament may be rewarded with an increase in their global user performance scores by 100, 50, and 25 for first, second and third place, respectively.

A user's tournament experience level provides an indication of their experience and success or failure in tournament play. Tournament experience levels start at a base level and can be earned or lost by a combination of one or more of standard athletic metric values, game-specific performance scores, user performance scores, and previous tournament performance earned or lost through screen-based and non-screen-based games.

Unlike a user's overall experience level, a user's tournament experience level may deteriorate to a lower level and/or may be capped at a maximum level. The deterioration of tournament experience levels may account for frequency of use, activity level, and/or previous tournament performance level. For example, if a user has not played any games or used the system 110 for an extended period, the user's tournament experience level may decrease and/or may be reset to zero.

The user's performance within a tournament can also result in the user's tournament experience level decreasing. For example, if a user enters a tournament and comes in the bottom 5% of players in the tournament, the user may lose a tournament experience level. The movement of tournament experience levels between finishing one tournament and entering the next one may be capped. For example, if a user comes in last place in a first tournament, their tournament experience level may decrease or increase by a maximum of 1 level before starting a second tournament.

Tournament experience levels may also be used to determine the types of tournaments that the user may enter. For example, a tournament may be defined to include an average tournament experience level to allow users to determine whether they should enter the tournament. For example, a tournament with an average ranking of 15-20 is appropriate for users who have a tournament experience level of 15-20 including a tolerance (e.g. +/−1). A user with a tournament experience level of 12 may enter the level 15-20 tournament but may have difficulty staying competitive with the other users.

Optionally, users may be blocked from competing in tournaments if they have an average tournament experience level ranking that is outside the specified range with tolerances. This may improve tournament competitiveness and prevent higher-level users from continually playing below their tournament experience level to gain a higher user performance score. Other methods to maintain competitiveness in tournaments may include reducing the tournament experience level of high-level players, reducing the user performance score achieved by high tournament experience level players, and/or adding penalties to high-level users playing in lower-level tournaments.

Optionally, higher average level tournaments may provide increased incentives (e.g. greater rewards) to encourage users to increase their tournament experience level and play in higher ranking tournaments. For example, a level 5-10 ranked tournament may allow the winner to increase their global user performance score by 50, and a 20-25 level tournament may allow the winner to increase their global user performance score by 1000.

In addition to tournaments, users may also participate in challenges. A challenge is a call to participate in a competition outside of regular game play or tournaments. Challenges may be shorter in length than tournaments. Challenges may be issued by other users or by the system. An increase in global user performance score or other rewards may be given to users who complete or win the challenge. For example, the system controller may issue a challenge of a 5-minute extra level in one of the games. A participant who completes the challenge would receive the challenge bonus (e.g. an increase in global user performance score by a certain amount). Another example may include a user challenging a friend to compete in one round of a dancing game. The winner of the challenge may receive an increase in their global user performance score.

Optionally, the user levels may include additional types of levels such as game-specific experience levels and game-specific tournament experience levels. The game-specific levels may be determined with the same formulas as the overall experience and tournament experience levels, except the game-specific levels are determined by the data from one game. The user may have more than one game-specific experience level and game-specific tournament experience level based on the user's participation in more than one game. For example, a user who plays a dancing game and a driving game may have separate game-specific experience levels and game-specific tournament experience levels for each game. The levels differ in value depending on the time spent on the activity, effort, and performance of a user for each game determined by one or more of standard athletic metric values, game-specific performance scores, and user performance scores earned while playing in each game, as well as global user performance scores, and previous tournament performance.

Game-specific tournament experience levels may not be offered for all games. Some games may not accommodate tournament play and therefore do not require tournament experience level assessments. Each game may have a different game-specific experience levelling framework. The game-specific levelling framework may result in a different number of levels, different thresholds to meet subsequent levels, and a different distribution of rewards to users. As a result, a user's game-specific experience level in one game after 25 hours of play may be different to their game-specific experience level for a second game played for the same amount of time.

The formulas used to determine overall experience levels and tournament experience levels, as well as game-specific experience levels and game-specific tournament experience levels may be displayed transparently for the user to view. Transparency of the level formulas may encourage users to perform activities in a manner that optimizes their level progression.

The system may provide a gaming user interface to a user of system 100. The gaming user interface can include a dashboard that displays user data and game-related data to the user. An example of a gaming user interface is shown in FIG. 4 and described in further detail herein below.

A user's progression towards a new overall experience level, tournament experience level, game-specific experience level, or game-specific tournament experience level may be displayed to the user in the dashboard. A graphic (e.g. a progression bar filling up with colour until the bar is full, when the new level is achieved) or numerical value (e.g. percentage) may be displayed to the user to communicate progression to a new level. Increasing user levels may also change the availability of rewards, with higher levels unlocking new rewards for the user to purchase. Increasing levels may also unlock special game play, special abilities, new digital items, new characters, and/or powers in games. Optionally, the rewards may be limited to certain types of playing experiences (e.g. single-player vs. multiplayer experiences). Increasing levels may also be used to unlock new game customizations, such as new songs and new skins.

Optionally, the system may also include a user currency system. The user currency system can allow users to accumulate user currency that can be redeemed for rewards and enhancements within the system and/or for specific gaming activities. User currency can be earned and traded for fungible rewards, non-fungible rewards, or physical rewards, using a virtual store within the system.

User currency can be acquired by users based on the accumulation of their global user performance scores. The user currency can be acquired by a user at an acquisition rate based on the global user performance score acquired by the user. For example, the user may acquire 0.1 dollars of user currency for every unit of their global user performance score. The ability to redeem user currency for rewards can further encourage users to continue to engage in physical activity while using the system.

The system may generate a rewards user interface that can be accessed by a user. The rewards user interface can display available rewards that can be obtained by redeeming user currency. The rewards user interface may be continually updated depending on the available options.

Each reward can be displayed with a description of the item and the amount of currency that must be redeemed to qualify for the reward. A user can select the reward they want and confirm that they have the amount of currency required. Then, the user may redeem the currency for the reward, and the required amount of user currency is removed from the user's account. The chosen reward is then sent to the user.

Examples of rewards may include NFTs (e.g. as is described in international patent application no. PCT/CA2022/050371, which is incorporated herein by reference in its entirety), skins and items for use in a game, easter eggs, screen effects, wall papers, other virtual goods, new levels, physical gifts (e.g. running shoes), discounts for goods and services, game advantages, congratulatory messages, cash, entries into draws for rewards, donations to a charity, and gift cards.

Optionally, unique user data for a user such as their standard athletic metric values or fitness level can be put on a blockchain and minted as an NFT. User currency may be spent in the virtual store of a specific game or may be spent in a system wide virtual store. When a reward is purchased by a user, the currency in the user's account is reduced while the global user performance score is unaffected.

Optionally, the system may distribute random or non-random user currency rewards to individuals or groups of users, as desired. For example, the system may award user currency gifts for signing up, starting new games, anniversaries, birthdays, milestones, encouraging return-to-play, etc.

Optionally, the system may track a user's fitness level. The user's fitness level may represent an estimate of the user's physiological fitness determined using the frequency, duration, and intensity of movements performed by the user (as determined from the sensor data). The user's fitness level may be determined from their standard athletic metric values.

Optionally, a user's fitness level may also integrate self-inputted data (e.g. nutrition) and may be linked to external applications (e.g. Apple™ health) and/or other sensorized devices (e.g. smartwatch) that also measure or record health data for determining an overall fitness level of the user.

A user's fitness level may be determined and modified over a specified period of time (e.g. daily, weekly, seasonally, etc.). For example, a user's fitness level may be calculated and updated daily at midnight. Unlike the overall and game-specific experience levels, fitness level is not a cumulative metric. Instead, the user receives a fitness level based on the sensor data, which can be compared to a scientific reference. For example, the sensor data may be correlated to VO2 Max to present fitness level information in a manner that is generally understood in the fitness community. The fitness level may be displayed to the user and tracked in the system and through the user's mobile application. The user may observe trends in fitness level data to understand how their fitness level is changing over time and make lifestyle changes to counteract any negative trends. Fitness levels may also be used to calculate recommended training loads for users.

Optionally, handicaps may also be applied to games to normalize user performance scores for novice users or users with physical characteristics that can negatively affect their performance. Handicaps can provide a proportional advantage to certain users to offset varying experience or competitor characteristics to equalize chances of success. This may encourage users to engage in movement activities with other users at different levels while still enjoying a competitive and engaging gaming or non-gaming experience.

Handicaps may be applied to game-specific performance scores and/or user performance scores. The handicaps can be applied to game-specific performance scores and user performance scores through the use of modification factors. For example, a modification factor may be applied to increase the performance score earned by a novice player, on lower difficulty or lower intensity movements compared to a more experienced competitor.

Handicaps can be applied manually or automatically, e.g. for competitions and tournaments. Handicaps may be automatically determined using one or more of recent tournament rankings, tournament experience levels, experience levels, game-specific tournament experience levels, game-specific experience levels, recent fitness levels, and recent system use-time (e.g. recent use of system 100) for each of the users. For example, handicaps may be automatically implemented on a sliding scale for novice players where greater handicaps are given to more novice players. For example, a group of four users playing a game can include one user who has never played before, one user who has played a few times, and two users who are high-level players. The users can elect to turn on the smart handicap function and proportional handicaps can be applied to the two less experienced users with no handicap for the two high-level users.

Optionally, the user performance score obtained by a user can be modified using modification factors based on specified criteria to encourage physical activity. A modification factor may be a value that is multiplied by the user performance score, or it may be a lump sum that is added to the user performance score. For example, modification factors can be applied to the formula for calculating user performance score to encourage physical activity. For example, performing a certain type of activity (e.g. running) under a certain set of conditions (e.g. minimum pace, minimum duration, etc.) can be used to trigger a modification factor to increase the user performance score during the activity. For example, a modification factor of 1.25× may be applied to the user performance score if users complete a run within 30 minutes of a notification. Modification factors may also be applied for reaching personal or group achievements.

A user's standard athletic metric values may trigger a modification factor or bonus for user performance scores and game-specific performance scores. For example, if a user achieves a milestone standard athletic metric value while playing a game, their game-specific performance score up to that point may double.

Modification factors may also be applied if activities are performed under specified conditions, such as challenging weather conditions, returning to the system after a prolonged absence, completing an extraordinary streak (e.g. 365 day streak), or wins/placement in a collaborative or competitive social event.

Modification factors may be applied for completing a group activity rather than an individual activity. For example, a modification factor could be applied for each additional user participating in a group activity. For example, if five users participate in a group run, their user performance scores during the run may increase 5× more than if they ran individually).

Modification factors may be applied if a user uses a partner product with the system. For example, if a running shoe company is a partner, a user's user performance score may be multiplied if a user goes for a run with the system 100, while wearing a pair of running shoes from the partner company.

Optionally, historical standard athletic metric values and/or game-specific performance scores can be stored by the system. This historical result data may be stored as a time series that can be retrieved for review and/or for use in a real-time activity. For example, a user may enable a “ghost” competition mode, where they can view their previous performances when repeating an activity, to compete against themselves. For example, in a game where a user is required to perform an explosive jump to break the ground underneath their avatar, they can view a “ghost” of their avatar's best performance while repeating the activity, along with a display window showing the ghost's instantaneous standard athletic metric value and game-specific performance score, to encourage them to match or improve the jump.

In some cases, highly-motivated users may put themselves at risk of injury or overexertion by engaging in strenuous or dangerous physical activity to increase their global user performance score. Optionally, the system may generate rewards for rest days to discourage users from overexerting themselves. For example, if an irregular exerciser suddenly embarks on a nine-day streak of strenuous activity to increase their global user performance score, they may receive a special reward on the tenth day if they take a rest. If users are concerned about breaking a streak on a rest day, the system may provide them with alternatives in order to maintain a streak. For example, if a user is on a nine-day streak of achieving a standard athletic metric value of 500 each day from running, on the tenth day the system may only require the user to achieve a standard athletic metric value of 100 from walking for the streak to be maintained. Rest rewards may be paired with third party integrations for specialized recovery. For example, a user may receive a special reward on a rest day if they perform a virtual stretch class with a third-party partner. The reward may be supplied by the third-party partner (e.g. a real or virtual yoga mat from a partner company).

Optionally, the system may specify a maximum value that global user performance scores can increase by on a given day or that user performance scores can increase by from a given activity, or a maximum number of days a streak can go on for. This may discourage users from overexerting themselves simply to increase their global user performance scores.

FIG. 4 illustrates an example graphical user interface that may be displayed to a user. FIG. 4 illustrates a graphical user interface showing a system dashboard that may be displayed to a user of system 100.

As discussed above, a user can interact with the system using a mobile application. For example, users can interact with the mobile application by selecting games to play, calibrating their insoles, viewing their stats, etc. The example dashboard shown in FIG. 4 is an example of a dashboard that can be displayed to the user to allow the user to interact with the system.

As shown in the example of FIG. 4, user data such as global user performance scores can be displayed on the dashboard. Standard athletic metric values, game-specific performance scores, user performance scores, and/or global user performance scores can be displayed as well.

The user data (e.g. the standard athletic metric values) can be displayed using various representations such as an average rate, a time-series rate, an accumulated value, historical values, and/or personal records. The dashboard may provide users with options to adjust the data that is visible and/or to view a breakdown/in-depth analysis of how standard athletic metric values, game-specific performance scores, user performance scores, and/or levels are achieved or calculated, for transparency to the user.

The dashboard may also include a leaderboard as in the example shown. The leaderboard may display the names and records of users with the highest global user performance scores, the highest overall experience level, and/or the highest game-specific level. Optionally, the leaderboard may also display tournament rankings.

The system may also interact with users, to provide them with feedback, notifications, and training goals. User data received by the system can be evaluated, monitored, and tracked over time to provide users with feedback and/or to generate goals for the user. For example, based on a user's standard athletic metric values over one week, a training goal can be generated for the user to achieve the same or greater standard athletic metric values the subsequent week.

Training goals can relate to an accumulated standard athletic metric value that should be achieved in a designated time period (session, day, week, month, year, season, etc.) or an instantaneous standard athletic metric value (i.e. a rate) that should be achieved at a certain point in time. Training goals can become increasingly targeted for individual users as additional sensor data is collected by the system over time.

Optionally, the system may allow users to accomplish their training goals by performing any type of activity, be it non-gaming or gaming. For example, if a user's weekly standard athletic metric goal is a value of 5000, they may reach their goal by achieving a standard athletic metric value of 3000 from an outdoor run, a standard athletic metric value of 1250 from a boxing game, and a standard athletic metric value of 750 from an adventure game.

Alternatively, the system may prescribe certain activities or games for the user to perform in order to meet their goals. For example, a user may be required to achieve a standard athletic metric value of 5000 from a non-gaming activity such as outdoor running. Training goals can be determined based on various types of user data such as athletic performance, system usage, athletic consistency, etc. Training goals can also be modified or created by the user.

Optionally, training goals may be generated and presented to users at pre-defined intervals (e.g. goals are presented daily and refreshed at 12:00 AM in the user's time zone).

The training goals can be defined to incentivize a user's physical activity. For example, the training goals may be defined to challenge users to increase their standard athletic metric values. Alternatively, a training goal may be defined to encourage users to decrease their standard athletic metric values if the obtained sensor data indicates that the user might be overexerting themselves or putting themselves at risk of injury.

Rewards can be provided to users for achieving their training goals. Optionally, based on a user's standard athletic metric values, the user's ability to perform a future activity can be predicted for a particular duration or distance (e.g. “It is estimated that the user can cover 5 km if their standard athletic metric value is 1000”).

The dashboard can also be used to communicate notifications and feedback to users. Notifications may include updates on user performance scores and standard athletic metric values, recent system use (or non-use), level progression, activity, other users, achievements, challenges, tournaments, and rewards.

Optionally, activity updates may use sensor data (e.g. standard athletic metric values) to prevent injuries, improve form, and/or help optimize performance during a game or non-game activity by notifying the individual of deviations from preferred behaviour or conformance to preferred behaviour. The notifications for activity updates may use “call-to-action” mechanics to specify what needs to be changed and when. Relevant metadata may be leveraged for the call-to action mechanics, depending on privacy preferences. For example, a user participating in a 10 km running game may have an extremely high standard athletic metric value in the 3^{rd} kilometre. The system may recognize the overexertion and notify the user that a slower pace or decrease in running intensity may be beneficial to overall performance. The system may also alert users to badges, streaks, discounts, or other achievements. Notifications may be communicated to the user via visual, audio, or haptic means. For example, when the sensor system reaches <10% battery, a vibration can be emitted to alert the user that the system needs to be charged.

During an activity, a user's standard athletic metric value, game-specific performance score, user performance score, and/or global user performance score can be displayed for the user in real-time, so that they are aware of their progress throughout the activity. For example, a user may glance at a mobile application on their phone or smartwatch while performing an activity such as a run to view their user performance score up to that point in the run. In another example, a portion (e.g. one corner) of a user's screen may be dedicated to displaying a user's standard athletic metric value, game-specific performance score, user performance score, and/or global user performance score when they play a game. Accordingly, the user can understand which movements or actions increase their metrics more than others in real-time, and they may adjust their performance to maximize their metrics. A summary screen may also be presented to a user at the end of their activity, which summarizes their metrics. Additionally, users may observe player statistics between active sessions. Player statistics may include hours played, last time played, past scores, games played, etc. over a set period and may be viewed on a page in the application.

Optionally, the system can include user authentication to ensure that only the specified user earns their standard athletic metric values, game-specific performance scores, user performance scores, and global user performance scores for their account (as opposed to friends, a machine, etc.). For example, a user can be authenticated using a gait identification process. The sensor data obtained at 310 can be used to evaluate the user's gait to determine that the gait data corresponds to the specified user.

Alternatively or in addition, other user authentication methods such as existing mobile-based user authentication techniques (e.g. facial recognition, fingerprint recognition, etc.) may be used to verify a user's identity.

Optionally, system 100 may also recommend specific activities to users. For example, specific activities may be assigned difficulty ratings (e.g. based on user RPE responses), and the system may recommend certain activities to users recovering from injuries based on the recovery of other users with similar injuries.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method of evaluating user performance across different activities using sensor data from a plurality of sensors positioned to contact a user, the method comprising:
obtaining sensor data from the plurality of sensors, wherein the sensor data is collected while the user performs one or more activities, the one or more activities comprising at least one of a gaming activity or a non-gaming activity;
for each activity of the one or more activities,
generating a standard athletic metric value from the sensor data corresponding to that activity;
determining an activity category of that activity; and
generating a user performance score from the standard athletic metric value and the activity category.

2. The method of claim 1, further comprising outputting the user performance score.

3. The method of claim 1, wherein the one or more activities comprises a plurality of activities and the method further comprises determining a global user performance score from the user performance scores generated for each of the activities in the plurality of activities.

4. The method of claim 3, further comprising determining a user experience level for the user based on the global user performance score.

5. The method of claim 1, wherein the plurality of sensors comprises a plurality of force sensors positionable underfoot of the user and the sensor data comprises force sensor data from the plurality of force sensors.

6. The method of claim 1, wherein the plurality of sensors comprises one or more IMUs mounted to the user and the sensor data comprises IMU data from the one or more IMUs.

7. The method of claim 1, wherein the standard athletic metric value comprises an energy expenditure value of the user.

8. The method of claim 1, wherein the standard athletic metric value comprises a rate of energy expenditure value of the user.

9. The method of claim 1, wherein the activity category is selected from the group consisting of a non-gaming activity, a screen-based gaming activity, and a non-screen-based gaming activity.

10. The method of claim 9, wherein the one or more activities comprises at least one non-gaming activity.

11. The method of claim 9, wherein the one or more activities comprises at least two of a non-gaming activity, a screen-based gaming activity, or a non-screen-based gaming activity.

12. The method of claim 1, wherein the one or more activities comprises a gaming activity and the method further comprises generating a game-specific performance score for the gaming activity from the sensor data associated with the gaming activity.

13. The method of claim 12, wherein the user performance score for the gaming activities is generated using the game-specific performance score.

14. The method of claim 1, wherein generating the user performance score comprises:
applying a function to the standard athletic metric value, wherein the function is defined according to the activity category.

15. The method of claim 14, wherein the function comprises a normalization factor that is applied to the standard athletic metric value.

16. The method of claim 14, wherein:
the one or more activities comprises a gaming activity;
a game-specific performance score is generated for the gaming activity from the sensor data associated with the gaming activity; and
the function defined for the gaming activity comprises a weighted sum of the game-specific performance score and the standard athletic metric value.

17. A system for evaluating user performance across different activities, the system comprising:
a plurality of sensors positionable to contact a user;
one or more processors communicatively coupled to the plurality of sensors;
wherein the one or more processors are configured to:
obtain sensor data from the plurality of sensors, wherein the sensor data is collected while the user performs one or more activities, the one or more activities comprising at least one of a gaming activity or a non-gaming activity;
for each activity of the one or more activities,
generate a standard athletic metric value from the sensor data corresponding to that activity;
determine an activity category of that activity; and
generate a user performance score from the standard athletic metric value and the activity category.

18. The system of claim 17, wherein the plurality of sensors comprises a plurality of force sensors positionable underfoot of the user and the sensor data comprises force sensor data from the plurality of force sensors.

19. The system of claim 17, wherein the plurality of sensors comprises one or more IMUs mounted to the user and the sensor data comprises IMU data from the one or more IMUs.

20. The system of claim 17, wherein the plurality of sensors are provided by one or more wearable devices worn by the user, and the wearable device is an insole.
